(19) United States
(12) Patent Application Publication
(10) Pub. No.: US 2008/0014199 A1
(43) Pub. Date: Jan. 17, 2008

(54) Assessing Cancer And Vimentin

(75) Inventors: Grzegorz S. Nowakowski, Rochester, MN (US); Neil E. Kay, St. Louis Park, MN (US); William G. Morice, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/753,767

(22) Filed: May 25, 2007

Related U.S. Application Data
(60) Provisional application No. 60/809,143, filed on May 25, 2006.

(51) Int. Cl. A61K 39/395; A61P 35/00; C12Q 1/02; C12Q 1/68

(52) U.S. Cl. 424/141.1; 435/29; 435/6

(57) ABSTRACT

This document provide methods and materials related to determining whether or not a mammal (e.g., a human) having cancer (e.g., leukemia) is susceptible to a poor outcome. For example, this document provides methods and materials involved in using the level of vimentin expression in leukemia cells from a mammal to determine whether or not the mammal is susceptible to a poor outcome.

### CROSS-REFERENCE TO RELATED APPLICATIONS

This application claims the benefit of U.S. Provisional Application Ser. No. 60/809,143, filed May 25, 2006.

### STATEMENT AS TO FEDERALLY SPONSORED RESEARCH

This invention was made with government support under grant CA095241 awarded by the National Cancer Institute of the National Institutes of Health. The government has certain rights in the invention.

### BACKGROUND

1. Technical Field

This document relates to methods and materials involved in assessing the prognosis of a mammal having cancer (e.g., leukemia). For example, this document provides methods and materials related to the use of vimentin expression levels to determine whether a mammal having cancer (e.g., chronic lymphocytic leukemia) is susceptible to a good or poor outcome.

2. Background Information

Chronic lymphocytic leukemia (CLL) is a lymphoproliferative disorder that can be characterized by clonal expansion of mature-appearing lymphocytes involving lymph nodes and other lymphoid tissues with progressive infiltration of bone marrow and presence in the peripheral blood. CLL accounts for about 40% of all leukemias in adults over the age of 65 years and is the most common leukemia in the United States. CLL increases in incidence with age and only rarely occurs in individuals less than 30 years of age. In some patients, CLL is a truly chronic leukemia, with survival of more than a decade. Other patients experience a rapid, lethal progression of the disease.

### SUMMARY

This document provides methods and materials related to determining whether or not a mammal (e.g., a human) having cancer (e.g., a leukemia such as CLL) has a poor prognosis. Some leukemia patients (e.g., CLL patients) experience a rapid cancer progression, and others experience an indolent disease for decades, frequently dying of unrelated causes (Chiorazzi et al. (2005) N. Engl. J. Med., 352(8):804-815). Identifying cancer patients (e.g., CLL patients) who have a poor prognosis can allow such patients, who are at risk for rapid progression, to be offered more aggressive therapy earlier. In addition, identifying cancer patients (e.g., CLL patients) who do not have a poor prognosis can avoid unnecessary treatment and patient suffering.

As described herein, differences in polypeptides expressed by cancer cells (e.g., CLL cells) with unmutated and mutated IgVH status were discovered using a phage display polypeptide library (PDPL). For example, unmutated CLL cells were observed to have significantly higher levels of vimentin expression than mutated CLL cells. In addition, a high level of vimentin expression in CLL cells was found to be a risk factor for a shorter time to initial treatment for patients with CLL.

In general, one aspect of this document features a method for assessing cancer. The method comprises, or consists essentially of, determining whether or not a mammal having lymphoid cancer, epithelial cancer, leukemia, or a lymphoproliferative disorder comprises cancer cells having an elevated level of expression of a vimentin polypeptide or mRNA encoding the vimentin polypeptide, wherein the presence of the elevated level indicates that the mammal is susceptible to a poor outcome. The mammal can be a human. The mammal can have leukemia. The leukemia can be chronic lymphocytic leukemia. The chronic lymphocytic leukemia can be early stage chronic lymphocytic leukemia. The cancer cells can be chronic lymphocytic leukemia cells. The chronic lymphocytic leukemia cells can be CD19 positive cells. The method can comprise determining whether or not the mammal comprises cancer cells having an elevated level of expression of the vimentin polypeptide. The method can comprise determining whether or not the mammal comprises cancer cells having an elevated level of expression of the mRNA encoding the vimentin polypeptide. The determining step can comprise fluorescence-activated cell sorting. The determining step can comprise PCR. The poor outcome can comprise a shorter time to treatment than the average time to treatment of a random population of 20 or more mammals having early stage cancer. The poor outcome can comprise a shorter survival time than the average survival time of a random population of 20 or more mammals having early stage cancer. The mammal can be treated if the mammal is susceptible to the poor outcome. The treatment can comprise a cancer treatment selected from the group consisting of chemotherapy, radiotherapy, splenectomy, leukapheresis, stem cell transplantation, and monoclonal antibody therapy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic diagram illustrating the subtraction biopanning procedure using a phage display polypeptide library and unmutated and mutated CLL B cells. The library containing phages displaying all pennutations of a 7-mer sequence is added to unmutated cells (UM; panel A). The phages displaying polypeptides that bind to unmutated cells are selected (panel B) and eluted (panel C). The eluted phages are then added to a suspension of mutated (M) cells (panel D). Phages bound to mutated cells are then removed, while unbound phages are amplified in E. coli culture (panel E). The procedure is repeated to enrich the pool of phages towards binding sequences.

FIG. 2 contains fluorescence photomicrographs showing binding of the FPSAHFL (SEQ ID NO:2) polypeptide to CLL B cells. CLL B cells were placed on slides by cytospining and were stained with phages displaying the FPSAHFL (SEQ ID NO:2) polypeptide and FITC labeled anti-phage antibody (A and D) or were stained directly with fluorescein conjugated FPSAHFL (SEQ ID NO:2) polypeptide (B, C, and E). Both the phage displaying the FPSAHFL (SEQ ID NO:2) polypeptide and directly fluorescently labeled FPSAHFL (SEQ ID NO:2) polypeptide localized within the cytoplasm (magnification 630×, objective 63×/1.32 oil, Leica Microsystems Digital Microscope DMRXA, Leica Microsystems, Bannockburn, Ill.; image acquisition: SenSys Photometrics KAF1400 camera, SenSyn Tucson, Ariz., and Leica QFISH V2.3 software, Leica Microsystems, Bannockburn, Ill.).

FIG. 3 contains fluorescence photomicrographs showing vimentin fibers visualized by immunofluorescence. Staining of cytospin CLL cells with FITC labeled anti-vimentin antibodies shows the presence of vimentin fibers in both unmutated (UM; panel A and B) and mutated (M; panel C and D) cells. Panels B and D were contrastained with DAPI. The vimentin cytoskeleton organization varies from diffuse (left panels) to polarized (right panels), resulting in apparent “capping.” The degree of polarization is variable form patient to patient, and in this example, “capping” is more prominent in mutated CLL cells (magnification 630×, objective 63×/1.32 oil, Leica Microsystems Digital Microscope DMRXA, Leica Microsystems; image acquisition: SenSys Photometrics KAF1400 camera, SenSyn and Leica QFISH V2.3 software, Leica Microsystems).

FIG. 4 is a graph plotting vimentin expression assessed by flow cytometry in 15 patients with mutated and unmutated IgVH. Vimentin expression is significantly higher in patients with unmutated IgVH (right) than mutated (left) IgVH.

FIG. 5 is a graph plotting Kaplan-Meier estimates of time to treatment (TTT) in months for patients with low and high vimentin expression. The median value of fluorescent intensity was used as a cutoff for risk stratification. The TTT in 40 patients with high versus low expression of vimentin was 2.8 and 12.9 years respectively, p=0.0025.

### DETAILED DESCRIPTION

This document provides methods and materials related to determining whether or not a mammal having cancer (e.g., lymphoid cancers (lymphomas), epithelial cancers, leukemia such as CLL, and other lymphoproliferative disorders) is susceptible to a poor outcome. For example, this document provides methods and materials for determining whether or not a vimentin expression level is elevated in cancer cells (e.g., CLL cells) from a mammal having cancer. As disclosed herein, if the level of vimentin expression in cancer cells (e.g., CLL cells) from a mammal is elevated, then the mammal can be classified as being susceptible to a poor outcome. If the level of vimentin expression in cancer cells (e.g., CLL cells) from a mammal is not elevated, then the mammal can be classified as not being susceptible to a poor outcome.

Any method can be used to identify a mammal as having cancer. For example, the diagnosis of CLL has usually been based on the criteria outlined by the International Workshop on Chronic Lymphocytic Leukemia (Ann. Intern. Med., 110(3):236-8 (1989)) and the National Cancer Institute-sponsored working group guidelines (Cheson et al., Am. J. Hematol., 29(3):152-63 (1988)). CLL can also be diagnosed based on an absolute peripheral blood lymphocytosis of greater than 5×10⁹/L together with the appearance of small mature looking lymphocytes, with an immunophenotype consistent with that described for CLL (Oscier et al., Br. J. Haematol., 125(3):294-317 (2004)). The typical immunophenotype consistent with CLL comprises CD19⁺ B cells which are also CD5⁺, CD23⁺, and FMC7 negative or very dim. The stage of CLL can be determined using any clinical staging system, such as the Rai staging system (Rai et al., Blood, 46(2):219-34 (1975) or the Binet staging system (Binet et al., Cancer, 48(1):198-206 (1981)).

In some cases, a mammal can be identified as having epithelial cancer by performing a histopathological examination of biopsy tissue, by using imaging techniques (e.g., CT, MRI, PET, FDG-PET coupled with MRI-CT, or PET/CT), or by applying a combination of such techniques. In some cases, a mammal can be identified as having lymphoma by performing a physical exam, blood tests, imaging tests (e.g., x-rays, CT scans, MRI, or a lymphangiogram), a biopsy, or any combination thereof.

Any mammal diagnosed as having cancer (e.g., CLL) can be assessed for susceptibility to a poor outcome. For example, a human, cat, dog, or horse having CLL can be assessed for susceptibility to a poor outcome. In some cases, a human with early stage CLL (e.g., Rai stage 0, 1, or 2) can be assessed. In some cases, a human between the ages of about 30-65 years old can be assessed. In some cases, a human older than about 60 years of age can be assessed. In some cases, a human less than about 40 years of age can be assessed.

The level of vimentin expression in cancer cells (e.g., CLL cells) from a mammal having cancer can be determined by measuring the vimentin polypeptide level in the cells. The vimentin polypeptide level can be determined by measuring the level of any vimentin polypeptide including, without limitation, a native, mutant, or variant vimentin polypeptide. Examples of vimentin polypeptides include, without limitation, human vimentin polypeptides (e.g., GenBank® accession number NP_{—}003371; gi|62414289), pig vimentin polypeptides (e.g., GenBank® accession number S05207; gi|418884), and mouse vimentin polypeptides (e.g., GenBank® accession number CAA39807; gi|55408).

In some cases, the level of vimentin expression in cancer cells (e.g., CLL cells) from a mammal having cancer can be determined by measuring the level of mRNA encoding a vimentin polypeptide in the cells. Examples of mRNA molecules encoding a vimentin polypeptide include, without limitation, mouse mRNA molecules encoding a vimentin polypeptide (e.g., GenBank® accession number X56397.1; gi|55407) and human mRNA molecules encoding a vimentin polypeptide (GenBank® accession number NM_{—}003380; gi|/62414288).

The term “elevated level” as used herein with respect to the level of vimentin expression is any level that is above a median vimentin expression level in cancer cells (e.g., CLL cells) from a random population of mammals (e.g., a random population of 10, 20, 30, 40, 50, 100, or 500 mammals) having early stage cancer (e.g., early stage CLL). In the case of CLL, an elevated level of vimentin expression can be any level of vimentin polypeptide expression in CLL cells that is determined as described herein and that results in a mean fluorescence intensity (MFI) for a vimentin polypeptide that is greater than 350, 375, 390, 400, 425, or 450. The level of vimentin expression can be characterized using variables or units other than MFI depending on the methods used to measure vimentin expression levels.

In some cases, an elevated level of vimentin expression can be a level of RNA encoding a vimentin polypeptide in cancer cells (e.g., CLL cells) from a mammal that is above a median level of RNA encoding a vimentin polypeptide in cancer cells (e.g., CLL cells) from a random population of mammals (e.g., a random population of 10, 20, 30, 40, 50, 100, or 500 mammals) of the same species having early stage cancer (e.g., early stage CLL). In some cases, an elevated level of vimentin expression can be any level of vimentin expression (e.g., vimentin polypeptide or vimentin RNA expression) that is above a median vimentin expression level in cancer cells from a population of mammals having early stage cancer that are of the same species, that are in the same age range (e.g., 30-65 years old, older than 60 years of age, less than 40 years of age, 25-40 years old, 40-50 years old, 50-60 years old, 60-70 years old, or 70-80 years old), that are of the same sex, and, in the case of humans, that are of the same race as the mammal being evaluated. In some cases, an elevated level of vimentin expression can be any level of vimentin expression (e.g., vimentin polypeptide or vimentin RNA expression) that is above a median vimentin expression level in cancer cells from a population of mammals that are of the same sex, that are in the same age range, and that have early stage cancer of the same stage (e.g., the same Rai stage).

It will be appreciated that vimentin expression levels from comparable samples of cancer cells (e.g., CLL cells) are used when determining whether or not a particular vimentin expression level is an elevated level. For example, a level of vimentin expression in CLL cells from a particular species of mammal is compared to the median level of vimentin expression in CLL cells from a population of mammals of the same species having early stage CLL. In addition, vimentin expression levels in cancer cells are compared to a median vimentin expression level measured using the same or a comparable method.

An elevated level of vimentin expression can be any level provided that the level is greater than a median vimentin expression level in cancer cells (e.g., CLL cells) from a random population of mammals (e.g., a random population of 10, 20, 30, 40, 50, 100, or 500 mammals) having early stage cancer (e.g., early stage CLL).

For example, an elevated level of vimentin expression can be 1.1, 1.2, 1.3, 1.4, 1.5, 1.6, 1.7, 1.8, 1.9, 2.0, 2.2, 2.4, 2.6, 2.8, 3.0, 3.3, 3.5, 3.7, 4.0, 4.5, 5.0, 6.1, 7.2, 8.0, 9.1, 10.0, 15.5, 20.7, or more times greater than a median vimentin expression level in cancer cells (e.g., CLL cells) from a random population of mammals (e.g., a random population of 10, 20, 30, 40, 50, 100, or 500 mammals) having early stage cancer (e.g., early stage CLL). In some cases, an elevated level of vimentin expression can be a level that is at least 2 percent (e.g., at least 3, 4, 5, 6, 7, 8, 9, 10, 15, 20, 25, 30, 35, 40, 45, 50, 55, 60, 65, 70, 75, 80, 85, 90, 95, 100, 200, 300, 400, or 500 percent) greater than a median vimentin expression level in cancer cells (e.g., CLL cells) from a random population of mammals (e.g., a random population of 10, 20, 30, 40, 50, 100, or 500 mammals) having early stage cancer (e.g., early stage CLL). In some cases, an elevated level of vimentin expression can be a level that is at least one standard deviation higher than a median vimentin expression level in cancer cells from a random population of mammals (e.g., a random population of 10, 20, 30, 40, 50, 100, or 500 mammals) having early stage cancer (e.g., early stage CLL).

Any method can be used to determine a vimentin expression level in cancer cells (e.g., CLL cells). For example, Northern blotting, RT-PCR, or quantitative PCR can be used to determine a level of mRNA molecules encoding a vimentin polypeptide in cancer cells. In some cases, mass spectrometry can be used to determine the level of vimentin polypeptides in cancer cells. In some cases, the level of vimentin polypeptides in cancer cells can be detected using a method that relies on an anti-vimentin antibody. Such methods include, without limitation, FACS, Western blotting, ELISA, immunohistochemistry, and immunoprecipitation. An anti-vimentin antibody can be labeled for detection. For example, an anti-vimentin antibody can be labeled with a radioactive molecule, a fluorescent molecule, or a bioluminescent molecule. Vimentin polypeptides can also be detected indirectly using a labeled antibody that binds to an anti-vimentin antibody that binds to vimentin polypeptides. An anti-vimentin antibody can bind to vimentin polypeptides at an affinity of at least 104 mol⁻¹ (e.g., at least 10⁵, 10⁶, 10⁷, 10⁸, 10⁹, 10¹⁰, 10¹¹, or 10¹² mol¹). Anti-vimentin antibodies are commercially available, e.g., from Santa Cruz Biotechnology, Santa Cruz, Calif.

The term “CLL cells” as used herein refers to mononuclear cells present in the blood of a mammal having CLL. CLL cells can be CD19⁺ cells. In some cases, CLL cells can be CD19⁺ and CD5⁺ cells. CLL cells present in the blood of a mammal having CLL can be analyzed for vimentin expression by obtaining a sample of blood from the mammal containing CLL cells and analyzing the CLL cells in the blood sample for vimentin expression.

For each of the methods and materials provided herein, a bone marrow sample or tumor biopsy sample can be used in place of a blood sample. Any method can be used to obtain a bone marrow sample or tumor biopsy sample including, without limitation, peripheral venipuncture, aspiration, core biopsy, and open surgical biopsy.

A blood sample can be obtained from a mammal by any method, such as peripheral venipuncture. The blood can be heparanized, and mononuclear cells, including CLL cells, can be isolated from the blood for analysis of vimentin expression. Any method can be used to isolate mononuclear cells from blood. For example, density gradient centrifugation can be used to isolate mononuclear cells. CCL cells can also be isolated from a blood sample for analysis of vimentin expression. Any method can be used to isolate CLL cells from a blood sample. For example, FACS can be used to isolate CLL cells from blood after labeling the cells with a fluorescent anti-CD19 antibody. In some cases, FACS can be used to isolate CLL cells from blood after labeling the cells with a fluorescent anti-CD19 antibody and a fluorescent anti-CD5 antibody. Such antibodies are commercially available, e.g., from BD Pharmingen, San Jose, Calif. Mononuclear cells or CLL cells isolated from a blood sample can be analyzed immediately for vimentin expression or cryopreserved and analyzed at a later time.

If mononuclear cells, rather than CLL cells, from a blood sample of a mammal having CLL are analyzed for vimentin expression, then the mononuclear cells can also be analyzed for expression of CD19 or for expression of CD19 and CD5 to determine the proportion of mononuclear cells that are CLL cells. The level of vimentin expression can be normalized to the proportion of CLL cells, e.g., by normalizing the level of vimentin expression to the level of CD19 expression or to the percentage of CLL cells in the mononuclear cell sample analyzed. In some cases, mononuclear cell samples can be labeled with more than one fluorescent-labeled antibody, e.g., a fluorescent-labeled anti-CD19 antibody and a fluorescent-labeled anti-vimentin antibody, where the antibodies are labeled with different fluorescent dyes that allow all of the antibodies to be detected simultaneously in the same sample. Such samples can be analyzed using, without limitation, FACS or immunohistochemistry.

An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-CD19 antibody or an anti-CD5 antibody can bind to CD19 polypeptides or CD5 polypeptides, respectively, at an affinity of at least 10⁴ mol⁻¹ (e.g., at least 10⁵, 10⁶, 10⁷, 10⁸, 10⁹, 10¹⁰, 10¹¹, or 10¹² mol⁻¹).

Once the level of vimentin expression in cancer cells (e.g., CLL cells) from a mammal is determined, then the level can be compared to a median level or a cutoff level and used to evaluate the susceptibility of the mammal to a poor outcome. A level of vimentin expression in CLL cells from a mammal that is higher than the median level of vimentin expression in CLL cells from a population of mammals of the same species having early stage CLL (or a cutoff level) can indicate that the mammal is susceptible to a poor outcome. In contrast, a level of vimentin expression in CLL cells from a mammal that is lower than the median level of vimentin expression in CLL cells from a population of mammals of the same species having early stage CLL (or a cutoff level) can indicate that the mammal is not susceptible to a poor outcome. A cutoff level can be set to any level provided that the values greater than that level correlate with an increased level of vimentin expression indicative of a mammal having an increased susceptibility to experience a poor outcome. For example, a cutoff level can be an MFI value of 390, 500, or 1200. In some cases, a cutoff level can be 5, 10, 15, 20, 25, 30, 35, 40, 45, 50, or 75 percent higher or lower than a median vimentin expression level in cancer cells (e.g., CLL cells) from a random population of mammals (e.g., a random population of 10, 20, 30, 40, 50, 100, or 500 mammals) having early stage cancer (e.g., early stage CLL). For example, a cutoff level can be 10 to 20 percent higher than a median vimentin expression level in cancer cells (e.g., CLL cells) from a random population of mammals (e.g., a random population of 10, 20, 30, 40, 50, 100, or 500 mammals) having early stage cancer (e.g., early stage CLL).

A mammal having early stage cancer (e.g., early stage CLL) that is susceptible to a poor outcome can require treatment for progressive disease sooner than a mammal having early stage cancer that is not susceptible to a poor outcome. For example, a mammal having early stage CLL that is susceptible to a poor outcome can require treatment for progressive disease within about 60 months from the initial diagnosis, e.g., within 1 month, 2 months, 3 months, 4 months, 5 months, 10 months, 15 months, 20 months, 30 months, 40 months, or 50 months from the initial diagnosis. A mammal having early stage CLL that is not susceptible to a poor outcome may not require treatment for more than 8 years, e.g., more than 9, 10, 11, 12, 13, 14, or 15 years, following the initial diagnosis.

In some cases, a mammal having early stage cancer (e.g., early stage CLL) that is susceptible to a poor outcome (e.g., that has cancer cells with an elevated level of vimentin expression) can experience a more rapid cancer progression than a mammal having early stage cancer that is not susceptible to a poor outcome (e.g., that does not have cancer cells with an elevated level of vimentin expression). In some cases, a mammal having early stage cancer (e.g., early stage CLL) that is susceptible to a poor outcome (e.g., has cancer cells with an elevated level of vimentin expression) can die sooner than a mammal having early stage cancer that is not susceptible to a poor outcome (e.g., that does not have cancer cells with an elevated level of vimentin expression).

The vimentin expression level can be used in combination with one or more other factors to determine whether or not a mammal having cancer (e.g., CLL) is susceptible to a poor outcome. For example, the vimentin expression level can be used in combination with the clinical stage; mutational status of IgVH; cytogenetic abnormalities, such as those detected by fluorescence in situ hybridization (FISH); lymphocyte doubling time; pattern of bone marrow infiltration on biopsy; serum beta-2-microglobulin level; thymidine kinase level; soluble CD23 level; percentage of CD38⁺ cells; or ZAP-70 expression level.

The susceptibility of a mammal having cancer (e.g., CLL) to a poor outcome can be determined over time to monitor disease progression. For example, a mammal having CLL can be assessed for susceptibility to a poor outcome at any time, particularly at the time of CLL diagnosis. The susceptibility of a mammal having cancer (e.g., CLL) to a poor outcome can be assessed once or more than once. Susceptibility to a poor outcome in cancer (e.g., CLL) can be assessed at regular intervals, e.g., every month, every other month, every four months, every six months, or every 12 months, or at irregular intervals, e.g., once a year for several years and then once a month. Susceptibility to a poor outcome in cancer (e.g., CLL) can be assessed during any time course, such during the course of a lifetime.

A mammal can be classified as having a cancer that has progressed if it is determined that the vimentin expression level in a sample containing cancer cells (e.g., a blood sample containing CLL cells) from the mammal is greater than the vimentin expression level in a corresponding sample obtained previously from the mammal. In some cases, a mammal can be classified as having cancer that has not progressed if it is determined that the vimentin expression level in a sample containing cancer cells (e.g., a blood sample containing CLL cells) from the mammal is equal to or less than the vimentin expression level in a corresponding sample obtained previously from the mammal.

Information about the susceptibility of a mammal having cancer (e.g., CLL) to a poor outcome can be used to guide treatment selection. For example, a mammal identified as having CLL and being susceptible to a poor outcome can be treated earlier and more aggressively than a mammal identified as having CLL and not being susceptible to a poor outcome. A more aggressive combined modality treatment can include chemo-immunotherapy followed by stem cell transplantation. A mammal identified as having CLL and not being susceptible to a poor outcome may undergo “watchful waiting” while having little or no standard treatment, particularly if the mammal is elderly. During this time, the susceptibility of the mammal to a poor outcome can be assessed periodically. When there is a change in the mammal's status, various treatment modalities can be used, such as chemotherapy, radiotherapy, splenectomy, leukapheresis, monoclonal antibodies and other immunomodulating agents.

A mammal that has been treated for cancer can be monitored for recurrence of the cancer. For example, a mammal that has been treated for cancer can be classified as having a recurring cancer if it is determined that a vimentin expression level in a sample (e.g., a blood sample) that would have contained cancer cells prior to treatment with the cancer therapy and that was obtained from the mammal after treatment is greater than the vimentin expression level observed in a corresponding sample (e.g., a blood sample) obtained from the mammal at an earlier time point after treatment. In some cases, a mammal can be classified as not having a recurring cancer if it is determined that a vimentin expression level in a sample (e.g., a blood sample) that would have contained cancer cells prior to treatment with the cancer therapy and that was obtained from the mammal after treatment is equal to or less than the vimentin expression level observed in a corresponding sample (e.g., a blood sample) obtained from the mammal at an earlier time point after treatment. A mammal can be monitored for recurrence of a cancer over any period of time with any frequency. For example, a mammal can be monitored once a year, twice a year, three times a year, or more frequently. In some cases, a mammal can be monitored every three months for five years, or once a year for as long as the mammal is alive.

Methods and materials provided herein also can be used to determine whether or not a cancer therapy is effective. For example, a vimentin expression level can be determined in a sample containing cancer cells (e.g., a blood sample) obtained from a mammal prior to treatment with a cancer therapy, and the level can be compared to the vimentin expression level in a corresponding sample (e.g., a blood sample) taken from a mammal during or after treatment. A decrease in the vimentin expression level in the sample taken during or after treatment as compared to the level in the sample taken before treatment can indicate that the treatment is effective. In some cases, an increase or no change in a vimentin expression level in a sample that would have contained cancer cells prior to treatment with a cancer therapy and that was obtained from a mammal during or after the treatment, as compared to the level in a corresponding sample taken before treatment, can indicate that the treatment is not effective. In some cases, a decrease in a level of vimentin expression in a sample that would have contained cancer cells prior to treatment with a cancer therapy and that was obtained from a mammal during or after a cancer treatment, as compared to the level in a corresponding sample taken at an earlier time point during treatment, can indicate that the treatment is effective. In some cases, an increase or no change in a level of vimentin expression in a sample that would have contained cancer cells prior to treatment with a cancer therapy and that was obtained from a mammal during or after a cancer treatment, as compared to the level in a corresponding sample taken at an earlier time point during treatment, can indicate that the treatment is not effective.

This document also provides methods and materials to assist medical or research professionals in determining whether or not a mammal having cancer is susceptible to a good or poor outcome. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of vimentin expression in a sample, and (2) communicating information about the level to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### Subtraction Biopanning Using a Phase Display Polypeptide Library

Blood was obtained from CLL patients who had provided written informed consent under a protocol approved by the Mayo Clinic Institutional Review Board according to the regulations of the Declaration of Helsinki. All CLL patients had a confirmed diagnosis based on the National Cancer Institute (NCI) Working Group definition (Cheson et al., Blood, 87(12):4990-7 (1996)). The patients encompassed all Rai stages and had not been treated for at least five weeks prior to collection of the blood samples. Mononuclear cells were isolated from heparinized venous blood by density gradient centrifuigation. For biopanning, immunoprecipitation (see, Example 3), and immunofluorescence (see, Example 2) experiments, samples were used that had more than 80% CLL B (CD5⁺CD19⁺) cells, as assessed by flow cytometry (FACScan; Becton Dickinson, Sunnyvale, Calif.). For other experiments, mononuclear cell samples were used regardless of the percentage of CD5⁺CD19⁺ cells. Mononuclear cells were either used less than 24 hours after being obtained from CLL patients or suspended in RPMI 1640 supplemented with 20% fetal calf serum and 10% dimethyl sulfoxide (DMSO) and stored at −80° C. until used. Clinical data including diagnosis, treatment, and survival were obtained from an existing prospective and actively maintained clinical database.

The IgVH mutational status was assessed as described elsewhere (Jelinek et al., Br J Haematol, 15(4):854-61 (2001)). The IgVH sequence in the patient samples was compared to the germ line sequence and was considered unmutated if the sequence identity was 98-100% and mutated if the sequence identity was less than 98%. For phage polypeptide library subtraction experiments, CLL B cell clones with 100% (unmutated) and less than 95% (mutated) homology to germ line were used to maximize potential differences in protein expression based on IgVH mutational status and improve phage subtraction specificity.

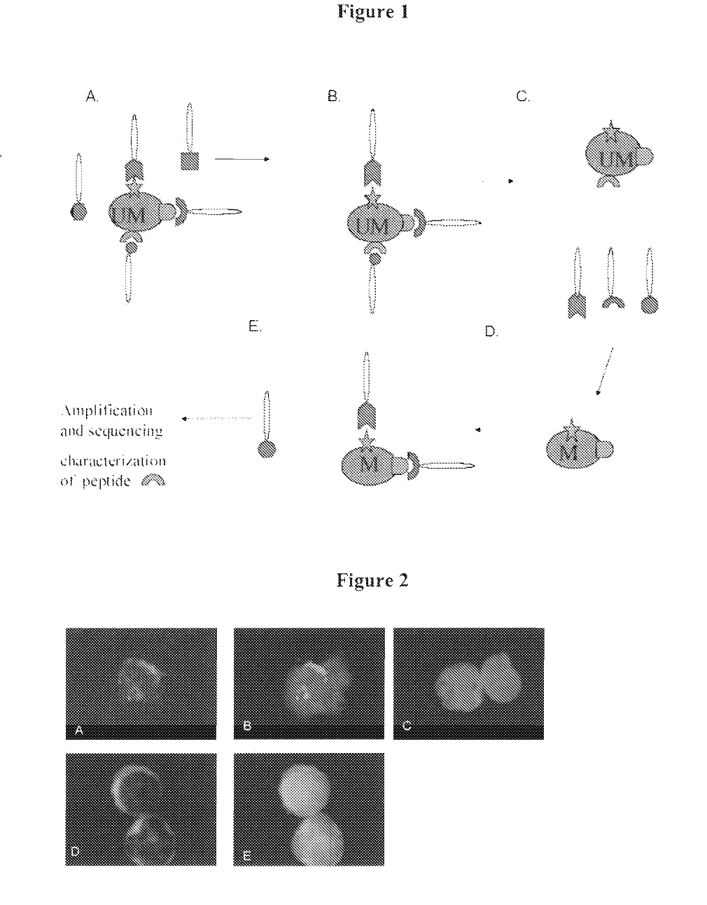

Phages displaying IgVH mutational status-specific polypeptides were isolated. 10¹¹ plaque forming units (pfu) of PDPL Ph.D.-7 (New England Biolabs, Ipswich, Mass.) were added to 10⁶ unmutated CLL B cells suspended in 1 mL of phosphate buffered saline (PBS) containing 0.5% bovine serum albumin (BSA), and the mixture was incubated at room temperature for 1 hour with gentle rocking (FIG. 1A). The cells with attached phages were spun down and washed three times with PBS containing 0.5% BSA (FIG. 1B). The attached phages were then eluted for 20 minutes by adding 1 mL of elution buffer (1% BSA, 0.2 M Glycine-HCl, pH 2.2; FIG. 1C). The elution buffer was neutralized with 0.15 mL of 1 M Tris-HCl (pH 9.1). The eluted phages were precipitated by adding 0.2 mL of a solution containing 20% (w/v) polyethylene glycol 8,000 (Sigma, Saint Louis, Mo.) and 2.5 M NaCl. The eluted phages were then added to a suspension of 10⁶ mutated CLL B cells in PBS with 0.5% BSA and incubated with gentle rocking for 40 minutes. The cells with bound phages (FIG. 1D) were spun down, leaving the supernatant containing unbound phages. This latter step was repeated four times to ensure complete subtraction of phages binding to mutated CLL B cells. The unbound phages in the supernatant were titered, amplified and purified as described elsewhere (Nowakowski et al., Stem Cells, 22(6):1030-8 (2004); FIG. 1E). The entire biopanning protocol was repeated three times, each time using newly amplified phages obtained from a previous round of subtraction biopanning in order to increase the specificity of the phage selection. Three rounds of biopanning with subtraction on mutated CLL cells yielded increasing phage titers: 3×10², 5×10⁴ and 3×10⁵ pfu/mL in the first, second, and third rounds of subtraction biopanning, respectively.

Phage sequencing was initiated after the third round of biopanning. Single phage colonies from the third round of subtraction biopanning, displaying polypeptides binding to unmutated cells, were amplified and sequenced as described elsewhere (Nowakowski et al., Stem Cells, 22(6):1030-8 (2004)). The corresponding polypeptide sequences were translated from phage DNA sequences using a reduced genetic code utilized by phage M13 according to the manufacturer's instructions.

Polypeptide sequences of amplified single phage colonies from the third round of biopanning with subtraction are presented in Table 1. The FPSAHFL sequence (SEQ ID NO:2) was of particular interest because it was encoded by three different phage colonies (20%) and was encoded by different phage DNA sequences. The finding of a single nucleotide polymorphism in an amino acid codon indicated that independent selection of the FPSAHFL (SEQ ID NO:2) polypeptide occurred in at least two different phage clones and isolation of the phages displaying the FPSAHFL (SEQ ID NO:2) polypeptide was unlikely to be the result of a random process. Sequencing of single phage colonies from the fourth round of biopanning revealed that the percentage of FPSAHFL (SEQ ID NO:2) polypeptide phage colonies had increased to 34%, indicating that further selection had occurred.

TABLE 1

Polypeptide sequences of single phage colonies
after three rounds of subtraction biopanning

| | |
|---|---|
| TCT ttt cct tcg gct cat ttt ctg GGT | SEQ ID NO:1 |
| F P S A H F L | SEQ ID NO:2 |
| TCT ttt cct tcg gct cat ttt ctt GGT | SEQ ID NO:3 |
| F P S A H F L | SEQ ID NO:2 |
| TCT ttt cct tcg gct cat ttt ctt GGT | SEQ ID NO:3 |
| F P S A H F L | SEQ ID NO:2 |
| TCT ctg tct tag aag cct tat cag GGT | SEQ ID NO:4 |
| L S Q K P Y Q | SEQ ID NO:5 |
| TCT aag cat act ctg ccg cat att GGT | SEQ ID NO:6 |
| K H T L P H I | SEQ ID NO:7 |
| TCT aat att tcg aat att agg ctt GGT | SEQ ID NO:8 |
| N I S N I R L | SEQ ID NO:9 |
| TCT gcg tat cct cgt guts cat aat GGT | SEQ ID NO:10 |
| A Y P R V H N | SEQ ID NO:11 |
| TCT aat ttg aag cct ctt tct atg GGT | SEQ ID NO:12 |
| N L K P L S M | SEQ ID NO:13 |
| TCT cat cct gtg tgg acg ctt ccg GGT | SEQ ID NO:14 |
| H P V W T L P | SEQ ID NO:15 |
| TCT tcg cat cct acg tct cat cat GGT | SEQ ID NO:16 |
| S H P T S H H | SEQ ID NO:17 |
| TCT cat gat ctg cct act cag cgt GGT | SEQ ID NO:18 |
| H D L P T Q R | SEQ ID NO:19 |
| TCT gct agt aat cat tat gag cgt GGT | SEQ ID NO:20 |
| A S N H Y E R | SEQ ID NO:21 |
| TCT gct gtt tcg gat cct cat agg GGT | SEQ ID NO:22 |
| A V S D P H R | SEQ ID NO:23 |
| TCT ctg cct gcg aat ttt cat ccg GGT | SEQ ID NO:24 |
| L P A N F H P | SEQ ID NO:25 |
| TCT gct ttg ccg act ctg cgt cag GGT | SEQ ID NO:26 |
| A V P T L R Q | SEQ ID NO:27 |

The single nuoleotide polymorphism in one of the codons is underlined.

### Example 2

### Cellular Localization of FPSAHFL (SEQ ID NO:2) Binding

Binding of the FPSAHFL (SEQ ID NO:2) polypeptide to CLL B cells was investigated using immunofluorescence. Cytospins of 10⁵ CLL B cells (see, Example 1) were prepared and incubated for 20 minutes at room temperature with phages displaying FPSAHFL (SEQ ID NO:2; 10⁸ pfu). The cells were then washed twice with PBS and incubated for 15 minutes at room temperature with 10 μg of mouse anti-M13 phage antibodies (New England Biolabs). The cells were stained for 15 minutes at room temperature with 2 μg of fluorescein isothiocyanate (FITC) labelled secondary anti-mouse antibodies (Vector Laboratories Inc, Burlingame, Calif., USA). Non-displaying M13 phages were used as a control (New England Biolabs) and co-staining with 4′-6-Diamidino-2-phenylindole (DAPI) was used to identify cells. Cytospin preparations of CLL B cells were also stained with fluorescein labelled FPSAHFL (SEQ ID NO:2) polypeptides. The heptapeptide was synthesized in the Mayo Clinic Proteomic Core Facility and labelled at the N terminus with fluorescein. The purity of the polypeptide preparation (>99.9%) was assessed by mass spectrometry and high performance liquid chromatography using internal standards.

A cytoplasmic pattern of staining was observed in CLL B cell cytospins incubated with phage and stained with fluorescent-labelled anti phage antibodies (FIG. 2A) and in CLL B cell cytospins stained with fluorescein-labelled FPSAHFL (SEQ ID NO:2) polypeptide (FIG. 2B).

### Example 3

### Identification of Vimentin as a Target of the FPSAHFL (SEQ ID NO:2) Polypeptide

To identify FPSAHFL (SEQ ID NO:2) polypeptide targets, CLL B cell lysates were immunoprecipitated using phage and anti-phage antibodies. Total protein extracts were prepared from primary leukemic cells (see, Example 1) by lysing cells in cold lysis buffer (30 mM Tris-HCl, pH 7.4; 150 mM NaCl; 1% glycerol (v/v); 1% Triton X-100 (v/v); and 2 mM EDTA) in the presence of phosphatase inhibitors (100 mM NaF, 10 mM Na₄P₂O₄, 1 mM Na₂VO₄), and protease inhibitors (10 μg/mL Leupeptin, 10 μg/mL Pepstatin A, and 1 mM phenylmethylsulfonyl fluoride (PMSF)). After centrifugation to remove cell debris, protein extracts were immunoprecipitated overnight at 4° C. on a rotator with phages displaying FPSAHFL (SEQ ID NO:2; 10¹¹ pfu, 6 hours), anti-M13 phage antibodies and 40 μL goat anti-mouse IgG-sepharose 4B beads (Zymed, San Francisco, Calif., USA), according to the manufacturer's instructions. The beads were washed three times with lysis buffer, resuspended into a loading buffer and subjected to SDS-PAGE (5%-15%) under reducing conditions. The polypeptide was visualized by Coomassie blue or silver staining. To confirm that the immunoprecipitated polypeptide was indeed the target for FPSAHFL (SEQ ID NO:2) phage, Western blotting was performed using staining with FPSAHFL (SEQ ID NO:2) phage and anti-M13 phage antibodies. Non-displaying phage were used as a control.

Immunoprecipitation of CLL polypeptides with phage bearing the FPSAHFL (SEQ ID NO:2) polypeptide and anti-phage antibodies identified a 56 kDa band. Binding of FPSAHFL (SEQ ID NO:2)-displaying phage to this band was confirmed by Western blotting. Another gel was run under the same conditions using the same immunoprecipitated polypeptide. A silver stained band observed at 56 kDa was excised and subjected to in-gel digestion. No other bands were observed at this molecular weight.

The immunoprecipitated polypeptide was identified by analyzing an in-gel trypsin digestion of the polypeptide sample using nano LC-MS/MS with linear ion trap mass spectrometry. Silver stained 1D gel spots were prepared for in-gel digestion using standard procedures. Spots were destained, reduced with DTT, and then alkylated with iodoacetamide. Polypeptides were digested with 3% trypsin (Promega Corporation, Madison, Wis.) in 20 mM Tris pH 8.1 at 37° C. for 12 hours followed by polypeptide extraction. Pooled extracts were concentrated and the volume was adjusted with 0.1% trifluoroacetic acid for polypeptide identification by nano-flow liquid chromatography tandem mass spectrometry (nano LC-MS/MS) analysis. The polypeptide mixture was trap injected onto a 75 μm×5 cm ProteoPep C18 PicoFrit nanoflow column and eluted with a 0.1% formic acid/acetonitrile gradient using a Michrom Paradigm MS4 (Michrom BioResources Inc., Auburn, Calif.) coupled to a ThermoFinnigan LTQ Linear Ion Trap mass spectrometer (ThermoElectron, San Jose, Calif.). Raw MS/MS data were converted to DTA files using ThermoFinnigan's Bioworks 3.1 and correlated to theoretical fragmentation patterns of tryptic polypeptide sequences from the NCBI nr and Swissprot databases using both SEQUEST™ and Mascot™ (Matrix Sciences, London, UK) search algorithms. Polypeptide identifications were considered when both Mascot and Sequest gave at least two consensus polypeptides with individual cross correlation or probability scores exceeding a threshold dependent on the precursor charge state, and ranking in the top five of all the hits for their respective MS/MS spectra. The target for the FPSAHFL (SEQ ID NO:2) polypeptide was identified as vimentin.

Mascot search results from polypeptide MS/MS spectra obtained from the digest showed that the highest scoring polypeptide was vimentin (Mascot Mowse Score: 1,611; M.W. 53,488 Da) with 15 tryptic polypeptides having independent scores greater than 50. The next highest scoring polypeptide identified in the search was actin (Mascot Mowse Score: 443; M.W. 41,710 Da) with only three tryptic polypeptides having independent scores greater than 50. These results strongly suggested that vimentin was indeed the polypeptide identified in the Western blot.

### Example 4

### Evaluation of Vimentin Expression as a Prognostic Factor in CLL

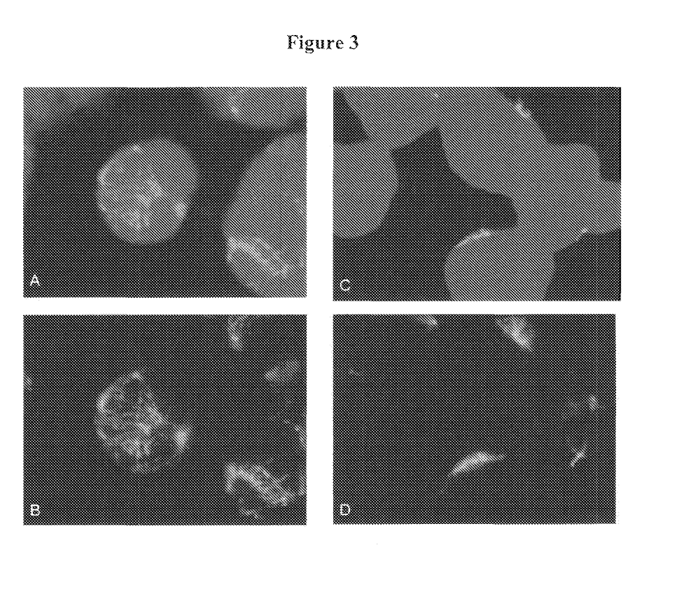

Analysis of cells using immunofluorescence indicated that vimentin fibres were present in both unmutated and mutated CLL B cells with variable degrees of polarization (capping; FIG. 3). To detect quantitative differences in vimentin expression between mutated and unmutated cells, vimentin fluorescence intensity was measured in CD19⁺ cells from the peripheral blood of patients with CLL using flow cytometry and gating on CD19⁺ cells.

Cryopreserved mononuclear cells from patients with CLL were defrosted and washed in RPMI. Vital cells (10⁶) were suspended in 0.1 mL of RPMI supplemented with 10% FCS. The cells were incubated with 10 μg of phycoerythrin (PE) labeled monoclonal anti-CD19 antibodies (BD Pharmingen, San Jose, Calif., USA) for 15 minutes at room temperature. The cells were washed with PBS, suspended in 0.2 mL of solution A (Fix and Perm Solution, Caltag Laboratories, Burlingame, Calif., USA) and incubated at room temperature for 15 minutes. The cells were then washed with PBS, suspended in solution B (Fix and Perm Solution, Caltag Laboratories) containing 2 μg of FITC labeled mouse anti-vimentin monoclonal antibodies (RV202; Santa Cruz Biotechnology, Santa Cruz, Calif.), and incubated for 15 minutes at room temperature. Cells were washed and analyzed by flow cytometry (FACScan; Becton Dickinson, Sunnyvale, Calif.). Isotype antibodies were used as controls. Expression of vimentin was assessed by gating on CD19⁺ cells (PE). The mean fluorescence intensity (MFI) of cells labelled with the vimentin antibody was compensated for the MFI of cells labelled with the isotype control antibody.

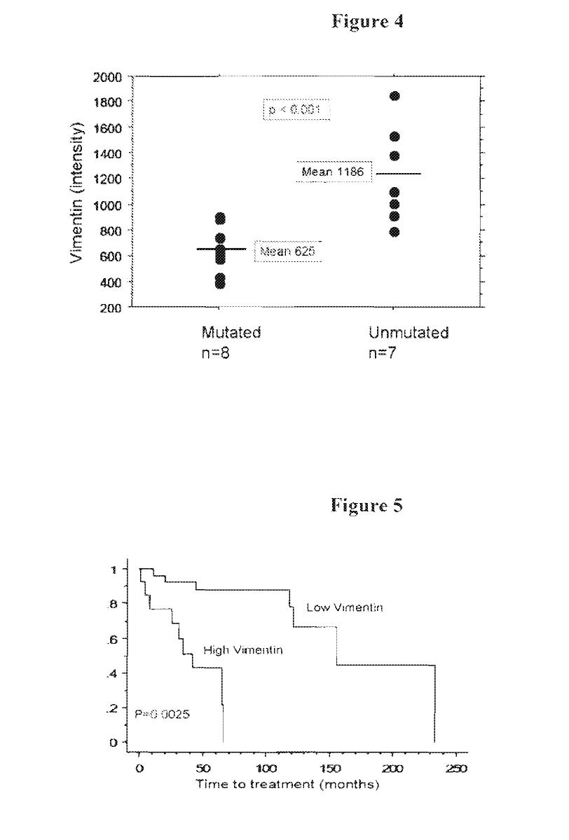

In an initial cohort of 15 CLL patients with known IgVH mutational status, vimentin expression was detectable in all patients, but was significantly higher in unmutated CLL B cells (MFI 1186; SD 381) than in mutated CLL B cells (MFI 625; SD109), p=0.001 (FIG. 4). MFI was used for subsequent comparisons, since vimentin was expressed at some level by CLL cells from all patients (MFI range of 311-1826).

To determine whether an association exists between vimentin expression and an adverse prognosis in CLL, vimentin expression was assessed using flow cytometry in cells from 40 patients with untreated, early stage CLL.

Archived samples with a median follow up of 81 months (range 40-233) were analyzed. CLL patient features are presented in Table 2. Mutational status was known for 37 patients. The MFI observed with unmutated CLL B cells was 512, while the MFI observed with mutated CLL B cells was 341 (p=0.023).

TABLE 2
CLL patient data

| | |
|---|---|
| Characteristic | N (%) |

Gender

| | |
|---|---|
| Male | 20 (50%) |
| Age | median 66, range 38-81 |

Rai stage

| | |
|---|---|
| 0 | 35 (87%) |
| I | 3 (8%) |
| II | 2 (5%) |

IgV_{H} mutation status

| | |
|---|---|
| Mutated | 20 (50%) |
| Unmutated | 17 (42%) |
| Unknown | 3 (8%) |

CD38 expression

| | |
|---|---|
| ≧30% | 10 (25%) |
| <30% | 30 (75%) |

Zap70 expression

| | |
|---|---|
| >20% | 10 (25%) |
| ≦20% | 7 (18%) |
| Unknown | 23 (57%) |

FISH

| | |
|---|---|
| Normal | 9 (23%) |
| 13q- | 9 (23%) |
| t12 | 1 (0.3%) |
| 11q- | 3 (1%) |
| 17p- | 2 (0.6%) |
| Other | 1 (0.3%) |
| Unknown | 15 (37%) |

Time to treatment (TTT) is an established surrogate marker of disease progression and overall survival in CLL (Rassenti et al., N. Engl. J. Med., 351(9):893-901 (2004); Calin et al., N. Engl. J. Med., 353(17):1793-1801 (2005)). TTT was defined as the time between the diagnosis of CLL and the initiation of treatment. TTT has been used as an endpoint for prognosis and as a surrogate for survival in CLL (Rassenti et al., N. Engl. J. Med., 351(9):893-901 (2004); Calin et al., N. Engl. J. Med., 353(17):1793-1801 (2005)). Patients who had not been treated were censored for purposes of TTT. TTT was estimated using the method of Kaplan and Meier, and the groups were compared using log-rank tests (Kaplan, Journal of American Statistic Association, 58:457-481 (1958)). Cox proportional hazards models were used to determine the impact of vimentin expression on TTT in univariate and multivariate analyses (Cox, Journal of the Royal Statistical Society, Series B, 34:187-220 (1972)). Comparisons between groups were performed using two-sample t-tests or rank sum tests for continuous variables and chi square tests for categorical variables. P-values less than 0.05 were considered to be statistically significant.

The MFI and TTT values for individual patients are presented in Table 3. The MFI of vimentin expression as a continuous variable was a risk factor for a shortened time to treatment (exponential coefficient 4.6, 95% confidence interval 1.33-15.22). Using the median MFI value in this patient cohort, patients were stratified into high and low vimentin expression groups. There was no difference in age, sex, or Rai stage distribution at diagnosis between patients having cells with vimentin expression levels above the median level and patients having cells with vimentin expression levels equal to or lower than the median vimentin expression level (p=0.43, p=0.65, and p=0.35, respectively). In contrast, the TTT differed significantly between patients having cells with high vimentin expression levels and patients having cells with low vimentin expression levels (2.8 and 0.9 years respectively, p=0.0025; FIG. 5). In univariate analysis, only high vimentin expression (greater than the median level) and CD38 expression (>30%) were associated with a shortened TTT, with hazard ratios of 4.67 (p=0.0064) and 7.5 (p=0.0008), respectively. However, Zap 70 expression, IgVH mutational status, and cytogenetics were available for only a limited number of patients (Table 2), likely limiting the predictive power of these factors. In multivariate analysis, only CD38 expression was an independent prognostic factor for shortened TTT (hazard ratio 1.4; p=0.0382). The number of patients was small, likely affecting the results of this analysis.

TABLE 3
Vimentin expression and time to treatment for
40 patients with archived blood specimens

| | Vimentin | TTT¹ |
|---|---|---|
| Patient | expression (MFI) | (months) |
| 1 | 409 | 56 |
| 2 | 434 | 56 |
| 3 | 531 | 31 |
| 4 | 547 | 42 |
| 5 | 315 | 122 |
| 6 | 278 | 63 |
| 7 | 239 | 52 |
| 8 | 383 | 77 |
| 9 | 482 | 34 |
| 10 | 353 | 175 |
| 11 | 1666 | 42 |
| 12 | 376 | 20 |
| 13 | 275 | 152 |
| 14 | 211 | 30 |
| 15 | 559 | 5 |
| 16 | 451 | 1 |
| 17 | 487 | 26 |
| 18 | 328 | 55 |
| 19 | 496 | 65 |
| 20 | 468 | 13 |
| 21 | 304 | 52 |
| 22 | 336 | 119 |
| 23 | 334 | 29 |
| 24 | 518 | 66 |
| 25 | 297 | 11 |
| 26 | 458 | 54 |
| 27 | 259 | 121 |
| 28 | 406 | 45 |
| 29 | 405 | 25 |
| 30 | 437 | 234 |
| 31 | 393 | 107 |
| 32 | 359 | 122 |
| 33 | 386 | 125 |
| 34 | 344 | 82 |
| 35 | 460 | 0 |
| 36 | 505 | 8 |
| 37 | 379 | 25 |
| 38 | 469 | 50 |
| 39 | 272 | 156 |
| 40 | 365 | 61 |

¹Time to treatment (TTT) was the time from original diagnosis to first treatment.

These results indicate that a high vimentin expression level in CLL cells is a risk factor for a shorter time to initial treatment for CLL patients.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing cancer, said method comprising determining whether or not a mammal having lymphoid cancer, epithelial cancer, leukemia, or a lymphoproliferative disorder comprises cancer cells having an elevated level of expression of a vimentin polypeptide or mRNA encoding said vimentin polypeptide, wherein the presence of said elevated level indicates that said mammal is susceptible to a poor outcome.

2. The method of claim 1, wherein said mammal is a human.

3. The method of claim 1, wherein said mammal has leukemia.

4. The method of claim 3, wherein said leukemia is chronic lymphocytic leukemia.

5. The method of claim 4, wherein said chronic lymphocytic leukemia is early stage chronic lymphocytic leukemia.

6. The method of claim 1, wherein said cancer cells are chronic lymphocytic leukemia cells.

7. The method of claim 6, wherein said chronic lymphocytic leukemia cells are CD19 positive cells.

8. The method of claim 1, wherein said method comprises determining whether or not said mammal comprises cancer cells having an elevated level of expression of said vimentin polypeptide.

9. The method of claim 1, wherein said method comprises determining whether or not said mammal comprises cancer cells having an elevated level of expression of said mRNA encoding said vimentin polypeptide.

10. The method of claim 1, wherein said determining step comprises fluorescence-activated cell sorting.

11. The method of claim 1, wherein said determining step comprises PCR.

12. The method of claim 1, wherein said poor outcome comprises a shorter time to treatment than the average time to treatment of a random population of 20 or more mammals having early stage cancer.

13. The method of claim 1, wherein said poor outcome comprises a shorter survival time than the average survival time of a random population of 20 or more mammals having early stage cancer.

14. The method of claim 1, wherein said mammal is treated if said mammal is susceptible to said poor outcome.

15. The method of claim 14, wherein said treatment comprises a cancer treatment selected from the group consisting of chemotherapy, radiotherapy, splenectomy, leukapheresis, stem cell transplantation, and monoclonal antibody therapy.


SEQUENCE LISTING (27 sequences)

SEQ ID NO: 1 - DNA, 27 bp, Artificial Sequence
  DNA sequence encoding polypeptide present in a phage library
tcttttccttcggctcattttctgggt

SEQ ID NO: 2 - protein, 7 aa, Artificial Sequence
  Polypeptide sequence present in a phage library
FPSAHFL

SEQ ID NO: 3 - DNA, 27 bp, Artificial Sequence
  DNA sequence encoding polypeptide present in a phage library
tcttttccttcggctcattttcttggt

SEQ ID NO: 4 - DNA, 27 bp, Artificial Sequence
  DNA sequence encoding polypeptide present in a phage library
tctctgtcttagaagccttatcagggt

SEQ ID NO: 5 - protein, 7 aa, Artificial Sequence
  Polypeptide sequence present in a phage library
LSQKPYQ

SEQ ID NO: 6 - DNA, 27 bp, Artificial Sequence
  DNA sequence encoding polypeptide present in a phage library
tctaagcatactctgccgcatattggt

SEQ ID NO: 7 - protein, 7 aa, Artificial Sequence
  Polypeptide sequence present in a phage library
KHTLPHI

SEQ ID NO: 8 - DNA, 27 bp, Artificial Sequence
  DNA sequence encoding polypeptide present in a phage library
tctaatatttcgaatattaggcttggt

SEQ ID NO: 9 - protein, 7 aa, Artificial Sequence
  Polypeptide sequence present in a phage library
NISNIRL

SEQ ID NO: 10 - DNA, 28 bp, Artificial Sequence
  DNA sequence encoding polypeptide present in a phage library
tctgcgtatcctcgtgutscataatggt

SEQ ID NO: 11 - protein, 7 aa, Artificial Sequence
  Polypeptide sequence present in a phage library
AYPRVHN

SEQ ID NO: 12 - DNA, 27 bp, Artificial Sequence
  DNA sequence encoding polypeptide present in a phage library
tctaatttgaagcctctttctatgggt

SEQ ID NO: 13 - protein, 7 aa, Artificial Sequence
  Polypeptide sequence present in a phage library
NLKPLSM

SEQ ID NO: 14 - DNA, 27 bp, Artificial Sequence
  DNA sequence encoding polypeptide present in a phage library
tctcatcctgtgtggacgcttccgggt

SEQ ID NO: 15 - protein, 7 aa, Artificial Sequence
  Polypeptide sequence present in a phage library
HPVWTLP

SEQ ID NO: 16 - DNA, 27 bp, Artificial Sequence
  DNA sequence encoding polypeptide present in a phage library
tcttcgcatcctacgtctcatcatggt

SEQ ID NO: 17 - protein, 7 aa, Artificial Sequence
  Polypeptide sequence present in a phage library
SHPTSHH

SEQ ID NO: 18 - DNA, 27 bp, Artificial Sequence
  DNA sequence encoding polypeptide present in a phage library
tctcatgatctgcctactcagcgtggt

SEQ ID NO: 19 - protein, 7 aa, Artificial Sequence
  Polypeptide sequence present in a phage library
HDLPTQR

SEQ ID NO: 20 - DNA, 27 bp, Artificial Sequence
  DNA sequence encoding polypeptide present in a phage library
tctgctagtaatcattatgagcgtggt

SEQ ID NO: 21 - protein, 7 aa, Artificial Sequence
  Polypeptide sequence present in a phage library
ASNHYER

SEQ ID NO: 22 - DNA, 27 bp, Artificial Sequence
  DNA sequence encoding polypeptide present in a phage library
tctgctgtttcggatcctcataggggt

SEQ ID NO: 23 - protein, 7 aa, Artificial Sequence
  Polypeptide sequence present in a phage library
AVSDPHR

SEQ ID NO: 24 - DNA, 27 bp, Artificial Sequence
  DNA sequence encoding polypeptide present in a phage library
tctctgcctgcgaattttcatccgggt

SEQ ID NO: 25 - protein, 7 aa, Artificial Sequence
  Polypeptide sequence present in a phage library
LPANFHP

SEQ ID NO: 26 - DNA, 27 bp, Artificial Sequence
  DNA sequence encoding polypeptide present in a phage library
tctgctttgccgactctgcgtcagggt

SEQ ID NO: 27 - protein, 7 aa, Artificial Sequence
  Polypeptide sequence present in a phage library
AVPTLRQ